(19) United States
(12) Patent Application Publication
(10) Pub. No.: US 2026/0240575 A1
(43) Pub. Date: Aug. 20, 2026

(54) ORTHOPEDIC IMPLANT FOR MULTI-FRAGMENTARY FRACTURE OF RADIAL HEAD OF RADIUS BONE AND METHOD OF IMPLANTING THE ORTHOPEDIC IMPLANT

(71) Applicant: Ritesh Arjunkumar RATHI, Ahmedabad (IN)

(72) Inventor: Ritesh Arjunkumar RATHI, Ahmedabad (IN)

(21) Appl. No.: 18/869,839

(22) PCT Filed: May 27, 2023
(86) PCT No.: PCT/IB2023/055456

§ 371 Date: Nov. 27, 2024

(30) Foreign Application Priority Data
May 27, 2022 (IN) 202221030559

(51) Int. Cl. A61B 17/72; A61B 17/00; A61B 17/56; A61F 2/38

(52) U.S. Cl. CPC A61B 17/7241; A61F 2/3804; A61B 2017/00477; A61B 2017/564; A61F 2002/3827

(57) ABSTRACT

An implant for a multi-fragmentary fracture of the radial head of the radius bone comprising: a truncated cone defined by an operative outer surface and an operative inner surface; configured with a plurality of first through-holes and a plurality of second through-holes along the wall of the truncated cone; a plurality of first fasteners configured to be received in said plurality of first through-holes to secure the arranged fragments of the multi-fragmentary fracture to operative outer surface of said cone and a nail defined by a tubular elongated body, said nail configured to operatively engage with said truncated cone to form said implant. The implant restores the native anatomy of the radial head and does not harm the functioning of surrounding tissues or nerves cell.

### FIELD

The invention relates to an orthopedic implant, especially for a multi-fragmentary fracture of the radial head of a radius bone, said implant comprising an implant body of generally circular shape configured to be in engagement with a nail having an elongated body received within a blind medullary canal of the radius bone.

The invention further relates to a method for implanting an implant for a multi-fragmentary fracture of a radial head of a radius bone, the method comprising the steps of:
extracting and arranging fragments of the multi-fragmental fracture of the radial head into the form of the restored radial head,
providing a template device in form of a ring having a bottom surface,
transferring the restored radial head to a template device configuration such that the top surface of the restored anatomy of the radial head is positioned towards the bottom surface of the template device;
providing a nail, said nail defined by an elongated body and a tip provided at the top end of the nail; and
reaming the portion of the medullary canal of the radius bone to predict the diameter of the nail.

### DEFINITIONS

As used in the present disclosure, the following terms are generally intended to have the meaning as set forth below, except to the extent that the context in which they are used indicates otherwise.

RADIAL HEAD: The term “RADIAL HEAD” hereinafter refers to a bone, which is the extreme part of the radius located near to the elbow, which is a disc-like structure.

MEDULLARY CANAL: The term “MEDULLARY CANAL” hereinafter refers to a central cavity of bone shafts where red bone marrow and/or yellow bone marrow are stored. The medullary canal has walls composed of spongy bone and is head with a thin, vascular membrane. The medullary canal is the area inside any bone that holds the bone marrow.

MULTI-FRAGMENT FRACTURE: The term “MULTI-FRAGMENT FRACTURE” hereinafter refers to a condition of bone fracture when the Radial head separates or has detached from the Radial bone and has several breaks in the Radial head, creating more than two fragments.

### BACKGROUND

The background information herein below relates to the present disclosure but is not necessarily prior art.

Typically, the fracture of the radius often occurs in the part of the bone near the elbow, called the radial head. Radial head fractures are common injuries, occurring in mostly acute elbow injuries.

There are varieties of surgical and non-surgical treatments available depending upon the severity of the injury. For example, surgical options for more severe injuries to the radial head can include open reduction with internal fixation (ORIF), radial head replacement (RHR), and total elbow replacement.

The ORIF is possible when radial head is fractured into three or less fragments. However, ORIF treatment induces significant stiffness in the elbow and limits the elbow functioning. Also, ORIF produces irritations to the surrounding soft tissue. Further, the RHR is not successful in long term usage. And the RHR process removes the radial head with a shaft attached disc like structure.

Further, Leibel DA et al. vide US patent titled “Radial head implant” described a radial head implant for replacement of a head portion of a radius bone. The implant includes a head, a stem, and a locking mechanism, where the head is adapted to engage with the stem. The implant head is adapted to unlock and disengage from the stem after engagement and locking. The stem being shaped to facilitate insertion of said stem into the medullary canal of the radius bone. The stem includes a collar positioned on a first portion of said stem to limit insertion of said stem into the medullary canal of the radius bone. However, the awarded patent is silent about the restoration of anatomy of radial head. Also, the awarded patent is silent about the alignment of the radial head with the radius bone.

Furthermore, Grisoni LT et al. vide US patent titled “Radial head implant system including modular implant and modular radial head locking instrument” described a modular radial head system including a modular implant for replacing the head of the proximal end of a radius and for articulating with the capitellum of a humerus. The modular implant includes a modular head and a modular stem. The system further includes a modular radial head locking instrument for locking the modular head and the modular stem of the modular implant to one another. The locking instrument includes a first jaw, a second jaw, and a control mechanism for urging the first and second jaws together to provide offset axial compression of the modular head and the modular stem of the modular implant. However, the awarded patent is also silent about the restoration of anatomy of radial head as well as about the alignment of the radial head with the radius bone.

Document US2021/0290402 published a semi-radial head prosthesis for use in the case that a partial articular surface of a radial head is fractured. The semi-radial head prosthesis comprises a prosthesis head matching to the radial head of the fractured 35 part. The prosthesis head is jointed with the fractured part by screws passing through fixing holes being arranged in the prosthesis in one plane and directed parallelly in one direction to the fractured part. An intact radial head is formed so that it is not necessary to remove the entire radial head. Instead, the radial head of the fractured part is replaced with a prosthesis head and the radial head of the non-fractured part is retained. This minimizes iatrogenic injuries to the patients, and is good for the joint intactness and joint stability. The prosthesis head has a hollow bone graft area for grafting fragments recessed on a side surface adjacent to the fractured surface. A recess for grafting fragments is disposed on a bottom side of the prosthesis head. The fragments are grafted in the hollow bone graft area are fragments of a fractured bone of the radial head. The prosthesis comprising screw fixing holes, adjacent to each other, each screw fixing hole having a central axis formed at an angle of 100-150 from a top surface of the non-fractured radial part. The prosthesis is not provided with any means for restoring the spatial anatomy of the radial head.

Document US2019/0254829 published an orthopedic implant apparatus comprising a body having a generally cylindrical shape comprising a concave upper surface and a flat bottom surface and a stem disposing in the longitudinal axis of the implant body and extending from the bottom surface of the body. The stem is inserted into the intramedullary canal of a radial bone. The concave upper surface of the body is adapted to support a spherical head of the opposite bone. The orthopedic implant is not provided with any means for restoring the spatial anatomy of the radial head.

Document US 2011/0112583 published a device for internal fixation of the bone fragments in a radius fracture. The device comprises at least one generally flat elongated body for abutment from outside against the fractured radial head of the bone and a number of fixing screws. The flat body is provided with fixing holes for a number of fixing screws passing through the flat body to the bone fragment in the radius fracture. The fixing screws having the axis being arranged in principle one plane in an obliquely crossed way. The body itself has in a distal end portion and in a proximal end portion at least two predrilled holes for fixing elements in form of a screw. To allow effective optimum internal fixation of the bone fragments in the radius fracture, the predrilled holes run obliquely relative to a longitudinal axis of the body so that all of the fixing elements abut against the bone fragments in plane. Each fixing element has at least one fixing portion for locking the fixing element in the bone fragment in the radius fracture and for locking the fixing element to the body. The fixing elements cross substantially the center part of the radius fracture and are disposed in principle in one longitudinal plane.

Therefore, there is a need for an implant which restores the anatomy of the radial head of the radius bone and eliminates the aforementioned drawbacks.

### OBJECTS

Some of the objects of the present disclosure, which at least one embodiment herein satisfies, are as follows:

An object of the present disclosure is to provide an implant for a multi-fragmentary fracture of the radial head of the radius bone.

Another object of the present disclosure is to provide an implant which restores the anatomy of the radial head of the radius bone.

Yet another object of the present disclosure is to provide an implant which enables the normal functioning of the elbow.

Yet another object of the present disclosure Is to provide an implant which provides flexibility to the joint.

Still another object of the present disclosure is to provide an implant which does not damage or disturb the functioning of surrounding tissues or nerve cells.

Yet another object of the present disclosure is to provide a method for assembling an implant for a multi-fragmentary fracture of the radial head of the radius bone for the purpose of implanting the implant.

Still another object of the present disclosure is to provide a method of implanting an implant in a medullary canal of the Radius bone.

Other objects and advantages of the present disclosure will be more apparent from the following description, which is not intended to limit the scope of the present disclosure.

### SUMMARY

The present disclosure envisages an implant for a multi-fragmentary fracture of the radial head of the radius bone. The implant comprises a truncated cone, a plurality of first fasteners, and a nail.

The drawbacks of the state of art are substantially eliminated and the aim of the invention is accomplished by the orthopedic implant, especially for a multi-fragmentary fracture of the radial head of a radius bone, said implant comprising an implant body of generally circular shape configured to be in engagement with a nail having an elongated body received within a blind medullary canal of the radius bone, according to the invention the essence of which consists in that
a template device is provided around the radial head of the radius bone to form the restored anatomy structure of the fragments of the multi-fragmental fracture of the radial head,
the implant body being a truncated cone having a sloping cone-shaped wall provided with a plurality of first through-holes along the wall, the bottom of the truncated cone provided with a central portion,
the nail having a tip being in engagement with the central portion of the bottom of the truncated cone,
a plurality of first fasteners passing the first through-holes in direction from the inner surface of the cone-shaped wall towards the outer surface of the truncated cone for securing the arranged fragments of the radial head to said truncated cone.

With advantage the top end of the nail may be configured to screw-fit or push-fit or snap-fit or glue-fit to the central portion of the truncated cone.

With advantage the tip provided at the top end of the nail being threaded is engaging with a threaded hole provided in the central portion of the truncated cone.

With advantage the inner surface of the central portion of truncated cone may be protruded.

With advantage the truncated cone may be provided in addition with a plurality of second through-holes along the wall and a plurality of second fasteners are provided to pass through the second through-holes in a direction from the outer surface 15 towards the inner surface of the truncated cone and being received within the cross-slits defined in the nail.

With advantage the nail may be provided with a plurality of cross-slits or with a plurality of teeth in form of tapered ribs around the circumference of nail.

With advantage the first fasteners and second fasteners may be of a type of bone screws selected from a group consisting of cannulated or non-cannulated screws, locking or non-locking screws, headless or headed screws, made from a biodegradable or a nonbiodegradable biocompatible material.

With advantage the truncated cone and the nail may be made from materials selected from a metal, or a biodegradable biocompatible material with advantage as calcium substitutes, or a non-biodegradable biocompatible material with advantage as plastics or a hybrid type.

The drawbacks of the state of art are substantially eliminated and the aim of the invention is accomplished further by a method for implanting an implant for a multi-fragmentary fracture of a radial head of a radius bone, the method comprising the following steps:
extracting and arranging fragments of the multi-fragmental fracture of the radial head into the form of the restored radial head,
providing a template device having a bottom surface,
transferring the restored radial head to a template device configuration such that the top surface of the restored anatomy of the radial head is positioned towards the bottom surface of the template device;
providing a nail, said nail defined by an elongated body and a tip provided at the top end of the nail;
reaming the portion of the medullary canal of the radius bone to predict the diameter of the nail; according to the invention consisting in
providing a truncated cone having a sloping cone-shaped wall provided with a plurality of first through-holes along the wall, the bottom of the truncated cone provided with a central portion,
reaming the restored anatomy of the radial head by using a reamer to create a space for truncated cone,
inserting the truncated cone into the created space;
providing a plurality of first fasteners;
inserting the first fasteners into the first through holes towards the outer surface of the truncated cone for securing the restored anatomy of the radial head to the truncated cone;
engaging the tip with the central portion of the bottom of the truncated cone,
inserting the nail in the proximal portion of the medullary canal of the radius bone.

With advantage following steps may be provided
providing a truncated cone having a sloping cone-shaped wall provided in addition with a plurality of second through-holes along the wall,
reaming the restored anatomy of the radial head by using a reamer to create a space for truncated cone,
inserting the truncated cone into the created space;
providing a plurality of first fasteners;
inserting the first fasteners into the first through holes (26) towards the outer surface of the truncated cone for securing the restored anatomy of the radial head to the truncated cone;
engaging the tip with the central portion of the bottom of the truncated cone,
providing a plurality of second fasteners,
inserting the second fasteners into the second through-holes to pass through the second through-holes in a direction from the outer surface towards the inner surface of the truncated cone and being received within the cross-slits defined in the nail,
inserting the nail in the proximal portion of the medullary canal of the radius bone.

With advantage the tip of the top end of the nail may be engaged with the central portion of the bottom of the truncated cone by screw-fitting or push-fitting or snap-fitting or glue-fitting.

The present disclosure envisages an implant for a multi fragmentary fracture of the radial head of the radius bone. The truncated cone is defined by a sloping wall, a recess, and a roof. The walls and the roof meet at a peripheral edge defining a circumferential peripheral edge; the wall and the roof defining an operative outer surface and an operative inner surface. The truncated cone is configured with a plurality of first through-holes along the wall. The plurality of first fasteners is configured to be received in the plurality of first through-holes. The first fastener secures the arranged fragments of the multi-fragmentary fracture of the radial head to the truncated cone.

In an embodiment, the truncated cone is configured with a plurality of second through-holes along the wall, wherein the diameter of the plurality of first through-holes being smaller than the plurality of second through-holes.

Further, the nail is defined by a hollow tubular elongated body. An operative top end of the nail is configured to operatively engage with an operative bottom of the truncated cone, to form the implant.

In an embodiment, the nail of the implant is configured to be received in a medullary canal of the radius bone.

The plurality of first fasteners is configured to pass through the plurality of first through-holes in a direction from the operative inner surface towards the operative outer surface of the truncated cone to secure the arranged fragments of the radial head to the operative outer surface of the truncated cone.

In an embodiment, the length of the first fastener is in the range of 6 mm to 30 mm.

In an embodiment, the diameter of the first fastener is in the range of 1.0 mm to 2.0 mm.

Further, the implant comprises a plurality of second fasteners. The plurality of second fasteners is configured to pass through the plurality of second through-holes in a direction from the operative outer surface to the operative inner surface of the truncated cone and further received within a plurality of cross-slits defined in the nail.

In an embodiment, the diameter of the second fastener is in the range of 1.5 mm to 3.5 mm.

In another embodiment, the length of the second fastener is in the range of 15 mm to 50 mm.

In another embodiment, the first fasteners and second fasteners are a type of bone screws selected from a group consisting of cannulated or non-cannulated screws, locking or nonlocking screws, or headless or headed screws, the fasteners made from a biodegradable or a non-biodegradable biocompatible material.

In another embodiment, the inner surface of the cone is configured with a central protruded portion where the central protruded portion is configured to operatively engage to the operative top end of the nail.

In another embodiment, the nail is a hollow tubular elongated body configured with a threaded tip at the operative top of the nail and the plurality of cross-slits, thereon.

In another embodiment, the nail is a hollow tubular elongated body with a threaded tip and a plurality of teeth defined by a plurality of ribs in a tapered configuration. Also, the nail is configured with the plurality of cross-slits, thereon.

In an embodiment, the operative top end of the nail is configured to screw-fit or push-fit or snap-fit or glue-fit (bone cement) to an operative outer surface of the central protruded portion.

In an embodiment, the truncated cone and the nail are made from materials selected from a metal, or a biodegradable biocompatible material such as calcium substitutes, or a non-biodegradable biocompatible material such as plastics or a hybrid type.

In an embodiment, the diameter of the nail is in the range of 6 mm to 10 mm depending on the diameter of the bone.

In an embodiment, the length of the nail is in the range of 20 mm to 60 mm.

Advantageously, the implant restores the anatomy of the radial head as well as does not harm or disturb the functioning of surrounding tissues or nerves cell.

Further, the present disclosure also envisages a method for implanting the implant for the multi-fragmentary fracture of the radial head in the medullary canal of the Radius bone. The implant is restoring the natural anatomy of the multi-fragmentary fracture Radial head of the radius bone. The method comprises the following steps:
extracting and arranging the multi-fragmental fracture of the radial head;
transferring the restored radial head to a template device in a configuration such that the operative top surface of the restored anatomy of the radial head moves towards the bottom surface of the template device;
reaming the proximal portion of the medullary canal of the radius bone to predict the diameter of a nail;
providing the nail, the nail is defined by the tubular elongated body;
providing the truncated cone, the truncated cone is defined by the sloping wall, the recess, and the roof, the wall and the roof defining the operative outer surface and the operative inner surface;
reaming the operative bottom of the restored anatomy of the radial head by using a reamer to create a space for the truncated cone of the implant;
operatively inserting said truncated cone in the created space;
providing the plurality of first fasteners;
inserting the plurality of first fasteners in to the plurality of first through-holes to secure the restored anatomy of the radial head to the truncated cone;
operatively engaging the inner surface of the truncated cone to an operative top end of nail to form the implant; and
inserting the nail in the proximal portion of the medullary canal of the Radius bone;
characterized in that:
providing with a plurality of first through-holes along the wall of the truncated cone; and
inserting the plurality of first fasteners in the plurality of first through-holes in a direction from the operative inner surface towards the outer surface of the truncated cone.

In another embodiment, the plurality of second fasteners are inserted to the plurality of second through-holes in a direction from the outer surface towards the operative inner surface of the truncated cone and further receiving within the plurality of cross-slits provided in the nail. The second fasteners are further protruding out from the nail.

In another embodiment, the inner surface of the cone has the central protruded portion where the central protruded portion is operatively engaging to the operative top end of the nail.

In an embodiment, allowing the operative top end of the nail to be push-fit or snap-fit or glue-fit (bone cement) with the operative outer surface of the central protruded portion.

Further, the method further includes inspecting the alignment of the restored anatomy of the radial head formed on the truncated cone with the radius bone, to thereby selecting the nail from a group consisting of the hollow tubular elongated body configured with a threaded tip, or the hollow tubular elongated body with a threaded tip and a plurality of teeth defined by a plurality of ribs in a tapered configuration around the circumference of the nail.

Advantageously, the implant is inserted inside the medullary canal of the Radius bone; therefore it minimizes the possibility of the elbow stiffness.

Further, the present disclosure also envisages a method for assembling the implant of the radius bone for the purposes of implanting the implant. The multi-fragment of the radial head is extracted and arranged. The method comprises the following steps:
transferring the restored anatomy of the radial head to the template device in a configuration such that the operative top surface of the restored anatomy of the radial head moves towards the bottom surface of the template device;
providing the nail, the nail is defined by an elongated body;
providing the truncated cone, the truncated cone is defined by the sloping wall, the recess, and the roof, the wall and the roof defining the operative outer surface and the operative inner
reaming the operative bottom of the restored anatomy of the radial head by using the reamer to create a space for the truncated cone of the implant;
operatively inserting the truncated cone in the created space;
providing the plurality of first fasteners;
inserting the plurality of first fasteners in to the plurality of first through-holes for securing the restored anatomy of the radial head to the truncated cone;
operatively engaging the inner surface of the truncated cone to the operative top end of the nail to form the implant; and characterized in that:
inserting the plurality of first fasteners in the plurality of first through-holes in a direction from the operative inner surface towards the operative outer surface of the truncated cone.

### BRIEF DESCRIPTION OF THE ACCOMPANYING DRAWING

The implant for a multi-fragmentary fracture of the radial head of the radius bone, assembling the implant and the method of implanting the implant, of the present disclosure, will now be described with the help of the accompanying drawing, in which:

FIG. 1 illustrates a multi-fragmentary fracture of the radial head of the Radius bone;

FIG. 2(A) and FIG. 2(B) illustrates a front view of an implant fitted inside the medullary canal of the radius bone in accordance with an embodiment of the present disclosure;

FIG. 3(A) and FIG. 3(B) illustrates an isometric view of a truncated cone with a plurality of first through holes, a plurality of second through holes and a central protruded portion in accordance with an embodiment of the present disclosure;

FIG. 3(C) illustrates an isometric view of a truncated cone with a plurality of first through holes and a plurality of second through holes in accordance with an embodiment of the present disclosure;

FIG. 4(A) illustrates a front view of a hollow tubular elongated nail and

FIG. 4(B) illustrates a front view of a hollow tubular elongated nail with a plurality of teeth defined by a plurality of ribs in a tapered configuration as per the embodiment of the present disclosure;

FIG. 5(A) illustrates an isometric view of the multi-fragmentary fracture of the radial head in accordance with an embodiment of the present disclosure;

FIG. 5(B) illustrates an isometric view of a template device with arranged fragments of the multi-fragmentary fracture of the radial head in accordance with an embodiment of the present disclosure;

FIG. 6 Illustrates an Isometric View of a Reamer Creating a space in the arranged fragments of the multi-fragmentary fracture of the radial head in accordance with an embodiment of the present disclosure;

FIG. 7 illustrates an isometric view of the fitments of the truncated cone and the assembly of the plurality of the first fasteners to secure fragments of the multi-fragmentary fracture of the radial head on the operative outer surface of the cone in accordance with an embodiment of the present disclosure;

FIG. 8 illustrates an isometric view of mounting the operative top end of the nail to the operative bottom of the truncated cone;

FIG. 9 illustrate a front view of the implant fitted inside the medullary canal of the radius bone in accordance with an embodiment of the present disclosure; and

FIG. 10 illustrates a cycle for implanting the implant for restoring the anatomy of the multi-fragmentary fracture of the radial head.

### LIST OF REFERENCE NUMERALS

20 multi-fragments fractured radial head
21 central protruded portion
22 truncated cone
23 opening
24 nail
25 teeth
25a threaded tip
26 first through-holes
28 second through-holes
30 inner surface of truncated cone
32 outer surface of truncated cone
34 first fasteners
36 second fasteners
38 cross-slits
42 top end of nail
44 bottom end of nail
46 template device
46a cap of template device
46b container of template device
48 reamer
50 radius bone
100 implant

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a”, “an”, and “the” may be intended to include the plural forms as well, unless the context clearly suggests otherwise. The terms “comprises”, “comprising”, “including”, and “having”, are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

When an element is referred to as being “mounted on”, “engaged to”, “connected to”, or “coupled to” another element, it may be directly on, engaged, connected or coupled to the other element. As used herein, the term “and/or” includes any and all combinations of one or more of the associated listed elements.

The terms first, second, third, etc., should not be construed to limit the scope of the present disclosure as the aforementioned terms may be only used to distinguish one element, component, region or section from another component, region, or section. Terms such as first, second, third etc., when used herein do not imply a specific sequence or order unless clearly suggested by the present disclosure.

Terms such as “inner”, “outer”, “beneath”, “below”, “lower”, “above”, “upper”, and the like, may be used in the present disclosure to describe relationships between different elements as depicted from the figures.

Typically, the open reduction with internal fixation (ORIF), is possible when radial head is fractured into three or less fragments. However, ORIF treatment induces significant stiffness in the elbow and limits the elbow functioning. Also, ORIF produces irritations to the surrounding soft tissue. Further, the radial head replacement (RHR) is not successful in long term usage. And the RHR process removes the radial head with a shaft attached disc like structure.

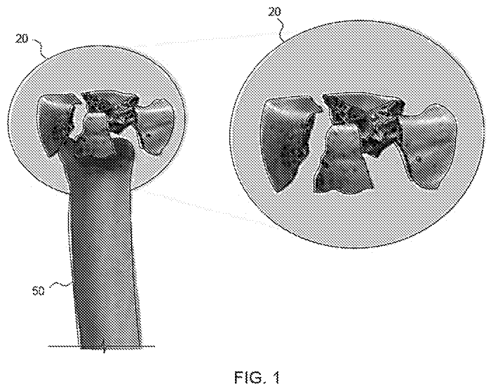

To overcome the aforementioned drawbacks, the present disclosure envisages an implant for a multi-fragmentary fracture of the radial head of the radius bone as shown in FIG. 1(a) and FIG. 1(b). The FIG. 1 illustrates a multi-fragmentary fracture of the radial head 20 of the Radius bone 50.

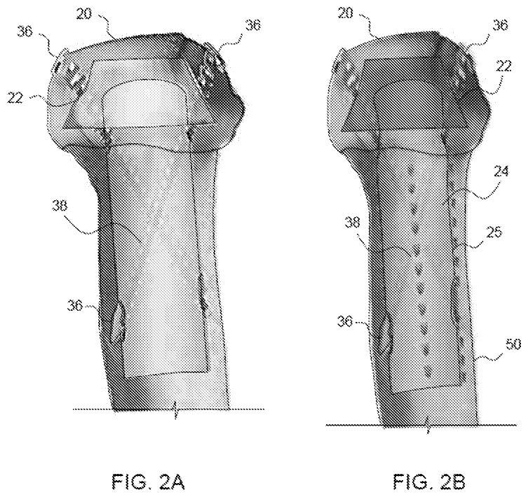

The implant 100 articulates the multi-fragmentary fracture of the radial head 20 and restores the anatomy of the radial head portion of the radius bone 50. However, the implant 100 of the present disclosure could be used for other joints with modifications to accommodate the particular size, anatomical shape and positioning, without changing the essential structure and operation of the proposed device of the present disclosure. FIG. 2(A) and FIG. 2(B) illustrates a front view of an implant fitted inside the medullary canal of the radius bone in accordance with an embodiment of the present disclosure.

The implant 100 of the present disclosure preserves the anatomy of the multi-fragmentary fracture of the radial head 20 of the radius bone 50. The implant 100 comprises a truncated cone 22, a plurality of first fasteners 34, and a nail 24.

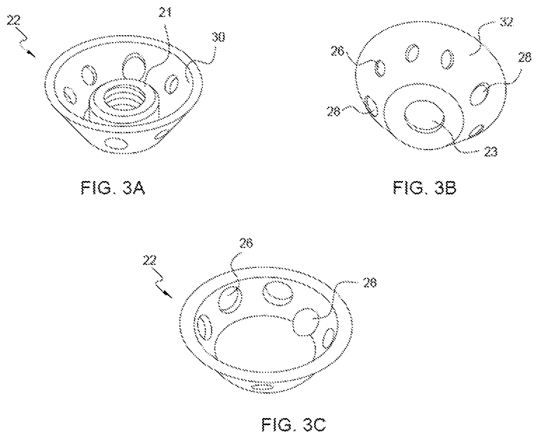

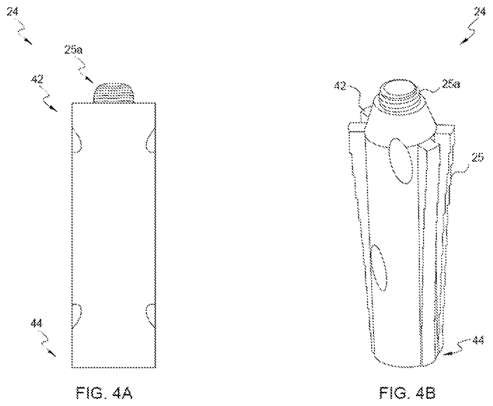

An embodiment of the present disclosure is described with reference to the FIG. 2(A) and FIG. 2(B), FIG. 3(A) and FIG. 3(B), FIG. 4(A) and FIG. 4(B). The truncated cone 22 of the implant 100 is shown in the FIG. 3(A), FIG. 3(B) and FIG. 3(C). The truncated cone 22 is defined by a sloping wall, a recess, and a roof. The walls and the roof meet at a peripheral edge defining a circumferential peripheral edge. Also, the wall and the roof defining an operative outer surface 32 and an operative inner surface 30. The truncated cone 22 is configured with a plurality of first through-holes 26 along the wall. The truncated cone 22 provides the structural support for the anatomy of the multi-fragmentary fracture of the radial head 20. The operative top end of the nail 24 is configured to operatively engage with an operative bottom of the truncated cone 22.

In an embodiment, the inner surface 30 of the truncated cone 22 is configured with a central protruded portion 21 where the central protruded portion 21 is configured to operatively engage to the operative top end 44 of the nail.

In an embodiment, the truncated cone 22 is configured with a plurality of second through-holes 28 along the wall, wherein the diameter of the plurality of first through-holes 26 is smaller than the plurality of second through-holes 28.

FIG. 3(A), FIG. 3(B) and FIG. 3(C) illustrate an isometric view of the truncated cone 22 with a plurality of first through holes 26 and a plurality of second through holes 28 in accordance with an embodiment of the present disclosure. FIG. 4(A) illustrates a front view of a hollow tubular elongated nail 24 with a threaded tip 25a and FIG. 4(B) illustrates a front view of the hollow tubular elongated nail 24 with a plurality of the teeth defined by the rib in a tapered configuration of the implant as per the embodiment of the present disclosure.

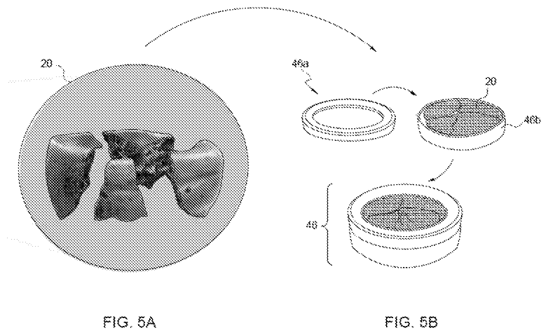

Further, the plurality of first fasteners 34 is provided and configured to be received in the plurality of first through-holes 26 of the truncated cone 22. The multi-fragments of the native radial head 20 are extracted and arranged to identify the size of the truncated cone 22 required. The multi-fragmentary of the radial head 20 are arranged or restored in a proper orientation to restore the native anatomy of the radial head. FIG. 5(A) illustrates an isometric view of the multi-fragmentary fracture of the radial head 20 in accordance with an embodiment of the present disclosure; The restored radial head is transferred to a template device 46 such that the operative top surface of the restored anatomy of the radial head moves towards the bottom surface of the template device 46 to form the anatomy of the multi-fragmentary fracture of the radial head 20. FIG. 5(B) illustrates an isometric view of the template device 46 with restored or arranged fragments of the multi-fragmentary fracture of the radial head 20 in accordance with an embodiment of the present disclosure. The operative bottom of the restored anatomy of the radial head is ream by using a reamer 48 to create a space for the truncated cone 22 of the implant 100.

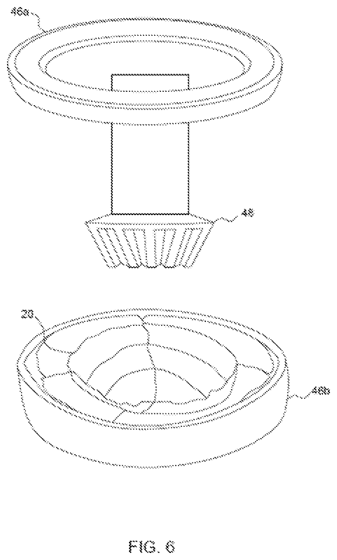

FIG. 6 illustrates an isometric view of the reamer 48 creating the space in the restored fragments of the radial head 20. The operative surface of the truncated cone 22 is inserted in the created space and the plurality of first fasteners 34 secures the fragments of the radial head 20 on the operative outer surface 32 of the truncated cone 22. Thereby, the first fastener 34 rigidly secures the fragments of the radial head 20 on the outer surface 32 of the truncated cone 22 and restores the near-net shape or anatomy of the radial head of the radius bone 50.

The plurality of first fasteners 34 is configured to pass through the plurality of first through-holes 26 in a direction from the operative inner surface 30 to the operative outer surface 32 of the truncated cone 22 to secure the fragments of the multi-fragmentary fractures of the radial head 20 thereon.

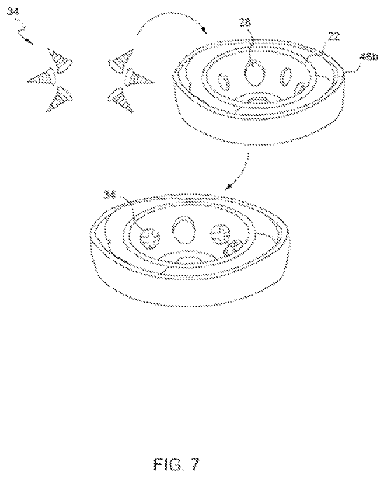

FIG. 7 illustrates isometric views of the fitments of the truncated cone 22 and the assembly of the plurality of the first fasteners 34 to secure the fragments of the radial head 20 on the operative outer surface 32 of the cone in accordance with an embodiment of the present disclosure.

In an embodiment, the plurality of first through-holes 26 is varying from 2 to 8 holes depending on the nature of fracture of the radial head.

In another embodiment, the plurality of second through-holes 28 is varying from 2 to 4 holes.

In an embodiment, the length of the first fastener 34 is in the range of 6 mm to 30 mm.

In another embodiment, the diameter of the first fastener 34 is in the range of 1.0 mm to 2.0 mm.

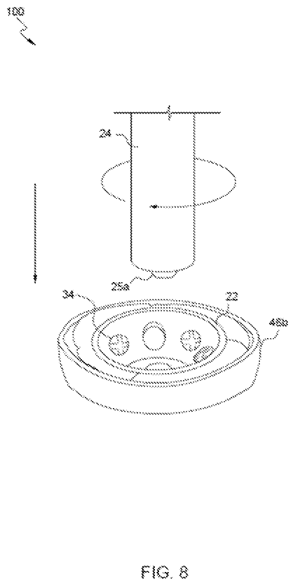

Further, the nail 24 is defined by an elongated body. The nail 24 is configured with a first end 42 and a second end 44, the first end 42 configured to be engaged with the operative bottom of the truncated cone 22. The truncated cone 22 is configured to be attached to the nail 24 in a fixed configuration so that the truncated cone 22 being restrained from rotational movement relative to the nail 24. The operatively engagement of the truncated cone 22 with the operative end of the nail 24, generates the required implant 100. The nail 24 of the implant 100 is configured to be received in a medullary canal of the radius bone 50. FIG. 8 illustrates an isometric view of mounting the operative top end of the nail 24 to the operative bottom of the truncated cone 22 in accordance with an embodiment of the present disclosure.

In an embodiment, the nail 24 is a hollow tubular elongated body configured with the threaded tip 25a on the operative top of the nail 24 and the plurality of cross-slits 38, thereon. In another embodiment, the nail 24 is a hollow tubular elongated body with a threaded tip 25a and a plurality of teeth 25 defined by a plurality of ribs in a tapered configuration around the circumference of the nail 24. Also, the nail 24 is configured with the plurality of cross-slits 38, thereon.

In another embodiment, the central protruded portion 21 is configured with a blind-threaded hole; the threaded tip 25a of the nail 24 is configured to be received within the blind-threaded hole.

In another embodiment, the central protruded portion 21 is configured with a through-threaded hole abutting the roof of the cone; the threaded tip 25a of the nail 24 is configured to be received within the through-threaded hole abutting the roof. In another embodiment, the operative top end 42 of the nail is configured to screw-fit or push-fit or snap-fit or glue-fit (bone cement) to an operative outer surface of the central protruded portion 21.

In another embodiment, the length of the nail 24 is in the range of 20 mm to 60 mm.

In another embodiment, a trial implant is used to predict the actual size, fit and alignment of the device 100.

In another embodiment, the implant 100 further may comprise a plurality of second fasteners 36. The second fasteners 36 are configured to secure the implant 100 within the medullary canal of the radius bone 50. The plurality of second fasteners 36 is configured to be received through the plurality of second through-holes 28 in a direction from the operative outer surface 32 to the operative inner surface 30 of the truncated cone 22 and further received within the plurality of cross-slits 38 defined in the nail 24. The second fasteners 36 are further configured to protrude out from the nail 24 to secure the nail 24 of the implant within the medullary canal of the Radius bone 50. The plurality of cross-slits 38 is configured to be in-line with the plurality of second through-holes 28 of the truncated cone 22.

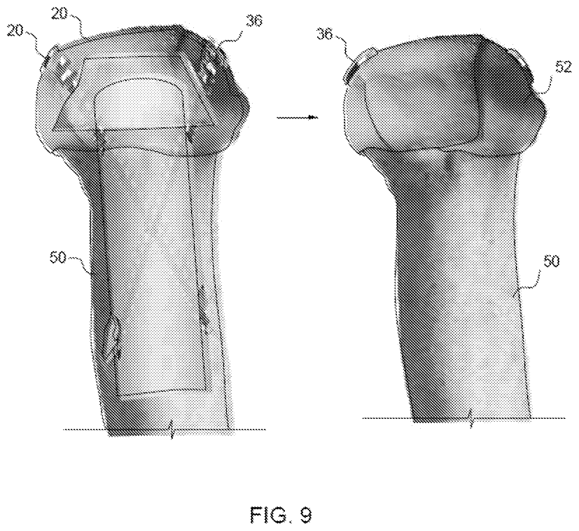

FIG. 9 illustrate a front view of the implant fitted inside the medullary canal of the radius bone 50 in accordance with an embodiment of the present disclosure.

In an embodiment, the diameter of the second fastener 36 is in the range of 1.5 mm to 3.5 mm.

In another embodiment, the length of the second fastener 36 is in the range of 15 mm to 50 mm.

In another embodiment, the diameter of the nail 36 is in the range of 6 mm to 10 mm. In an embodiment, the plurality of cross-slits 38 is varying from 2 to 4, and preferably limited to 2 cross-slits.

Further, the first fasteners 34 and the second fasteners 36 are a type of bone screws selected from a group consisting of a cannulated or non-cannulated screw, a locking or non-locking screw, or a headless or headed screw, said fasteners 34, 36 made from a biodegradable or a non-biodegradable biocompatible material. Also, the truncated cone 22 and the nail 24 are made from materials selected from a metal, or a biodegradable biocompatible material such as calcium substitutes, or a non-biodegradable biocompatible material such as plastics or a hybrid type.

Advantageously, the implant 100 of the present disclosure preserves the native anatomy of the radial head as well as does not damage or disturb the functioning of surrounding tissues or nerve cells.

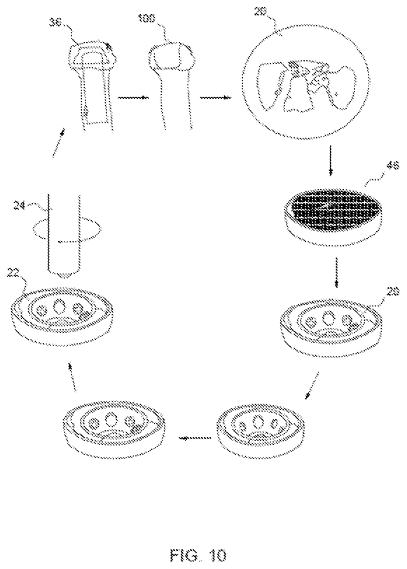

Further, the present disclosure also envisages a method for implanting the implant 100 in a medullary canal of the radius bone 50. FIG. 10 illustrates a cycle for implanting the 30 implant for restoring the anatomy of the multi-fragmentary fracture of the radial head. The implant 100 is restoring the natural anatomy of the multi-fragmentary fracture of the radial head 20. The method comprises the following steps:
extracting and arranging the multi-fragments fracture of the radial head 20;
transferring the restored anatomy of the radial head to the template device 46 in a configuration such that the operative top surface of the restored anatomy of the radial head moves towards the bottom surface of the template device 46;
reaming the proximal portion of the medullary canal of the radius bone 50 to predict the diameter of the nail 24;
providing the nail 24, the nail is defined by an elongated body;
providing the truncated cone 22, the truncated cone is defined by the sloping wall, the recess, and the roof, the wall and the roof defining the operative outer surface 32 and the operative inner surface 30;
reaming the operative bottom of the restored anatomy of the radial head by using the reamer 48 to create a space for the truncated cone 22 of the implant 100;
operatively inserting the truncated cone 22 in the created space;
providing the plurality of first fasteners 34;
inserting the plurality of first fasteners 34 in to the plurality of first through-holes 26 for securing the restored anatomy of the radial head to the truncated cone 22;
operatively engaging the inner surface of the truncated cone 22 to the operative top end of the nail 24 to form the implant 100; and
inserting the nail 24 in the proximal portion of the medullary canal of the Radius bone 50; characterized in that:
providing a plurality of first through-holes 26 along the wall of the truncated cone 22; and
inserting the plurality of first fasteners 34 in the plurality of first through-holes 26 in a direction from the operative inner surface 30 towards the operative outer surface 32 of the truncated cone 22.

Further, the plurality of first fasteners 34 are inserted into the plurality of first through-holes 26 in a direction from the operative inner surface 30 towards the outer surface 32 of the truncated cone 22. The plurality of second fasteners 36 are inserted into the plurality of second through-holes 28 in a direction from the operative outer surface 32 towards the operative inner surface 30 of the truncated cone 22 and further allowed to be received within the plurality of cross-slits provided in the nail. The second fasteners 36 are further protruding out from the nail for securing the nail 24 of the implant within the medullary canal of the radius bone.

In an embodiment, the anatomy of the multi-fragmentary fracture of the Radial head of the radius bone 50 is formed in the template device 46.

Further, the present disclosure also envisages a method for assembling the implant 100 of the radius bone 50 for the purposes of implanting the implant. The multi-fragment of the radial head is extracted and arranged. The implant 100 is restoring the natural anatomy of the multi-fragmentary fracture of the radial head 20. The method comprises the following steps:
transferring the restored anatomy of the radial head to the template device 46 in a configuration such that the operative top surface of the restored anatomy of the radial head moves towards the bottom surface of the template device 46;
providing the nail 24, the nail is defined by an elongated body;
providing the truncated cone 22, the truncated cone is defined by the sloping wall, the recess, and the roof, the wall and the roof defining the operative outer surface 32 and the operative inner surface 30;
reaming the operative bottom of the restored anatomy of the radial head by using the reamer 48 to create a space for the truncated cone 22 of the implant 100;
operatively inserting the truncated cone 22 in the created space;
providing the plurality of first fasteners 34;
inserting the plurality of first fasteners 34 in to the plurality of first through-holes 26 for securing the restored anatomy of the radial head to the truncated cone 22; and
operatively engaging the inner surface of the truncated cone 22 to the operative top end of the nail 24 to form the implant 100;
characterized in that:
inserting the plurality of first fasteners 34 in the plurality of first through-holes 26 in a direction from the operative inner surface 30 towards the operative outer surface 32 of the truncated cone 22.

The method further includes inspecting the alignment of the restored anatomy of the radial head formed on the truncated cone 22 with the radius bone 50, to thereby selecting the nail 24 from a group consisting of the hollow tubular elongated body configured with the threaded tip 25a, or the hollow tubular elongated body with the threaded tip 25a at the operative top of said nail 24 and a the plurality of cross-slits 38 defined, thereon, and the plurality of teeth 25 defined by a plurality of ribs in a tapered configuration around the circumference of the nail 24 as shown in the FIG. 2(a) and FIG. 2(b) and the FIG. 4(a) and FIG. 4(b).

Advantageously, the implanting of the implant 100 is inside the medullary canal, therefore it avoids and minimizes the possibility of the elbow stiffness and provides enough flexibility for the movement of the arms.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

### Technical Advances and Economical Significance

The present disclosure described herein above has several technical advantages including, but not limited to, the realization of the implant for the multi-fractured radial head of the radius bone, assembling the implant, and the method of implanting the implant, that:
preserve the native radial head of the radius;
eliminates the uncertainty of the radial head replacement (RHR);
provides flexibility to the joint;
avoids the possibility of bone stiffness due to extra medullary implant;
implant remains inside the medullary canal; and
does not damage or harm the functioning of surrounding tissues or nerve cells.

The foregoing description of the specific embodiments so fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the of the disclosure and not as a limitation.

1.-22. (canceled)

23. An orthopedic implant, especially for a multi-fragmentary fracture of the radial head of a radius bone, said implant comprising an implant body of generally circular shape configured to be in engagement with a nail having an elongated body received within a blind medullary canal of the radius bone, comprising:
a template device is provided around the radial head of the radius bone to form the restored anatomy structure of the fragments of the multi-fragmental fracture of the radial head,
the implant body being a truncated cone having a sloping cone-shaped wall provided with a plurality of first through-holes along the wall, the bottom of the truncated cone provided with a central portion,
the nail having a tip being in engagement with the central portion of the bottom of the truncated cone, and
a plurality of first fasteners passing the first through-holes in direction from the inner surface of the cone-shaped wall towards the outer surface of the truncated cone for securing the arranged fragments of the radial head to said truncated cone.

24. The implant according to claim 23, wherein
the top end of the nail is configured to screw-fit or push-fit or snap-fit or glue-fit to the central portion of the truncated cone.

25. The implant according to claim 23, wherein
the tip provided at the top end of the nail being threaded is engaging with a threaded hole provided in the central portion of the truncated cone.

26. The implant according to claim 23, wherein the inner surface of the central portion of truncated cone is protruded.

27. The implant according to claim 23, wherein the truncated cone is provided in addition with a plurality of second through-holes along the wall and a plurality of second fasteners are provided to pass through the second through-holes in a direction from the outer surface towards the inner surface of the truncated cone and being received within the cross-slits defined in the nail.

28. The implant according to claim 27, wherein the nail is provided with a plurality of cross-slits or with a plurality of teeth in form of tapered ribs around the circumference of nail.

29. The implant according to claim 27, wherein the first fasteners and second fasteners are of a type of bone screws selected from a group consisting of cannulated or non-cannulated screws, locking or non-locking screws, headless or headed screws, made from a biodegradable or a nonbiodegradable biocompatible material.

30. The implant according to claim 23, wherein the truncated cone and the nail are made from materials selected from a metal, or a biodegradable biocompatible material with advantage as calcium substitutes, or a non-biodegradable biocompatible material with advantage as plastics or a hybrid type.

31. A method for implanting an implant for a multi-fragmentary fracture of a radial head of a radius bone, the method comprising the following steps:
extracting and arranging fragments of the multi-fragmental fracture of the radial head into the form of the restored radial head,
providing a template device having a bottom surface,
transferring the restored radial head to a template device configuration such that the top surface of the restored anatomy of the radial head is positioned towards the bottom surface of the template device;
providing a nail, said nail defined by an elongated body and a tip provided at the top end of the nail; and
reaming the portion of the medullary canal of the radius bone to predict the diameter of the nail;
providing a truncated cone having a sloping cone-shaped wall provided with a plurality of first through-holes along the wall, the bottom of the truncated cone provided with a central portion,
reaming the restored anatomy of the radial head by using a reamer to create a space for truncated cone,
inserting the truncated cone into the created space;
providing a plurality of first fasteners;
inserting the first fasteners into the first through holes towards the outer surface of the truncated cone for securing the restored anatomy of the radial head to the truncated cone;
engaging the tip with the central portion of the bottom of the truncated cone, and
inserting the nail in the proximal portion of the medullary canal of the radius bone.

32. The method according to claim 31, further comprising:
providing a truncated cone having a sloping cone-shaped wall provided in addition with a plurality of second through-holes along the wall,
reaming the restored anatomy of the radial head by using a reamer to create a space for truncated cone,
inserting the truncated cone into the created space;
providing a plurality of first fasteners;
inserting the first fasteners into the first through holes towards the outer surface of the truncated cone for securing the restored anatomy of the radial head to the truncated cone;
engaging the tip with the central portion of the bottom of the truncated cone,
providing a plurality of second fasteners,
inserting the second fasteners into the second through-holes to pass through the second through-holes in a direction from the outer surface towards the inner surface of the truncated cone and being received within the cross-slits defined in the nail, and
inserting the nail in the proximal portion of the medullary canal of the radius bone.

33. The method according to claim 31, further comprising:
engaging the tip of the top end of the nail with the central portion of the bottom of the truncated cone by screw-fitting or push-fitting or snap-fitting or glue-fitting.

34. The implant according to claim 24, wherein the inner surface of the central portion of truncated cone is protruded.

35. The implant according to claim 25, wherein the inner surface of the central portion of truncated cone is protruded.

36. The implant according to claim 24, wherein the truncated cone is provided in addition with a plurality of second through-holes along the wall and a plurality of second fasteners are provided to pass through the second through-holes in a direction from the outer surface towards the inner surface of the truncated cone and being received within the cross-slits defined in the nail.

37. The implant according to claim 25, wherein the truncated cone is provided in addition with a plurality of second through-holes along the wall and a plurality of second fasteners are provided to pass through the second through-holes in a direction from the outer surface towards the inner surface of the truncated cone and being received within the cross-slits defined in the nail.

38. The implant according to claim 26, wherein the truncated cone is provided in addition with a plurality of second through-holes along the wall and a plurality of second fasteners are provided to pass through the second through-holes in a direction from the outer surface towards the inner surface of the truncated cone and being received within the cross-slits defined in the nail.

39. The implant according to claim 24, wherein the truncated cone and the nail are made from materials selected from a metal, or a biodegradable biocompatible material with advantage as calcium substitutes, or a non-biodegradable biocompatible material with advantage as plastics or a hybrid type.

40. The implant according to claim 25, wherein the truncated cone and the nail are made from materials selected from a metal, or a biodegradable biocompatible material with advantage as calcium substitutes, or a non-biodegradable biocompatible material with advantage as plastics or a hybrid type.

41. The implant according to claim 26, wherein the truncated cone and the nail are made from materials selected from a metal, or a biodegradable biocompatible material with advantage as calcium substitutes, or a non-biodegradable biocompatible material with advantage as plastics or a hybrid type.

42. The implant according to claim 27, wherein the truncated cone and the nail are made from materials selected from a metal, or a biodegradable biocompatible material with advantage as calcium substitutes, or a non-biodegradable biocompatible material with advantage as plastics or a hybrid type.
